(19) United States
(12) Patent Application Publication
(10) Pub. No.: US 2026/0096960 A1
(43) Pub. Date: Apr. 9, 2026

(54) Anhydrous Powder Hair Lightening Suspension

(71) Applicant: Henkel AG & Co. KGaA, Duesseldorf (DE)

(72) Inventor: David Erlingheuser, Wallingford, CT (US)

(21) Appl. No.: 19/352,434

(22) Filed: Oct. 7, 2025

Related U.S. Application Data
(63) Continuation of application No. PCT/US2024/050225, filed on Oct. 7, 2024.

(51) Int. Cl. A61K 8/23; A61K 8/25; A61K 8/31; A61Q 5/08

(52) U.S. Cl. CPC A61K 8/23; A61K 8/25; A61K 8/31; A61Q 5/08; A61K 2800/30

(57) ABSTRACT

A crème composition for lightening keratin fibers that includes about 5 to about 60% by weight of a non-volatile oil component, about 3 to about 30% by weight petrolatum, about 0.05 to about 5% by weight fumed silica, about 1 to about 70% by weight of a persulfate salt, and 0% by weight microplastics.

### TECHNICAL FIELD OF THE INVENTION

The present disclosure relates to agents for oxidative color changes in the field of cosmetics which are particularly suitable for lightening keratin fibers, in particular human hair.

### BACKGROUND OF THE INVENTION

Hair bleaching involves the application of an oxidizing agent such as a hydrogen peroxide solution to the hair for a period of time effective to achieve a desired lighter hair shade. The addition of a persulfate salt compositions is used to enhance the bleaching effect of the hydrogen peroxide solution. Prior approaches to hair bleaching involved mixing a persulfate salt and alkaline agents in the form of a powder with the hydrogen peroxide solution prior to use to form a mixture that is applied to the hair for a desired period of time result in a lightening of the hair color. Other approaches involved mixing the powder with a liquid to form a paste prior to application to the hair. The powder component has the disadvantage that when mixed with the hydrogen peroxide solution or other liquid, it can aerosolize. The airborne powder may be breathed in by the consumer resulting in an unpleasant experience.

Anhydrous powder hair lightening suspensions (i.e. crème hair lighteners) are desired by consumers because the compositions eliminate the aerosolization of solids associated with powder hair lighteners when mixed with hydrogen peroxide solutions (i.e. developers). Formulations were developed to overcome the problem with aerosolization by providing the consumer with a mixture of the powder components in mineral oil. However, early mixtures were found to be unstable, with phase separation of the oil and the salt phases. Attempts to use waxes or stearates also did not work as the resulting formulations are waxy and get hard and dry (solidification) after 6-9 months. Block polymers have been used to give mineral oil powder suspension benefits in anhydrous composition and a rheology profile to allow the composition to be conveniently dispensed from a tube. However, block polymers are microplastics which have detrimental ecological impact to aquatic life. Furthermore, high temperatures are required to process the block polymers into hair lightening suspension formulations.

Microplastics are a particular environmental burden in our oceans. Positive steps have been taken to reduce microplastics load in the environment. The United States banned plastic microbeads in cosmetics and personal care products through the Microbead-Free Waters Act of 2015. The Act defines the term “plastic microbead” to mean any solid plastic particle that is less than five millimeters in size and is intended to be used to exfoliate or cleanse the human body or any part thereof. Banning plastic microbeads only eliminates one category of microplastics pollution.

Thus, there is a need in the art for crème hair lightener formulations that provide powder suspension benefits to an anhydrous mineral oil and desired rheology profile without the use of microplastics.

### BRIEF SUMMARY OF THE INVENTION

Disclosed, in various embodiments, are crème compositions for lightening keratin fibers, comprising about 5 to about 60% by weight of a non-volatile oil component, about 3 to about 30% by weight petrolatum, about 0.05 to about 5% by weight fumed silica, about 1 to about 70% by weight of a persulfate salt, and 0% by weight microplastics, wherein all amounts are based on the total weight of the crème composition.

These and other features and characteristics are more particularly described below.

### DETAILED DESCRIPTION OF THE INVENTION

The following detailed description is merely exemplary in nature and is not intended to limit the disclosure or the application and uses of the subject matter as described herein. Furthermore, there is no intention to be bound by any theory presented in the preceding background or the following detailed description.

Disclosed are crème compositions for lightening keratin fibers, the composition comprising an anhydrous gelling composition and powder components and is free of microplastics. The anhydrous gelling composition comprises a non-volatile oil component, petrolatum, and fumed silica. Notably, no microplastics are present in the anhydrous gelling composition. The anhydrous gelling composition provides homogeneous suspension benefits to powder components such as persulfate salts (oxidizer), alkalizers, and modifiers (fillers, viscosity enhancers, chelating agents). As the crème composition is free of microplastics, particularly free of block polymers used in the art to aid in the suspension of powder components, it is ecofriendly. In addition, the processing conditions used to prepare the microplastics-free crème composition have a lower carbon footprint due to the lower heating requirements versus typical block polymer-based formulations. Thus, the microplastics-free crème composition has a preferred sustainability profile.

Petrolatum controls the adsorption of the non-volatile oil component (e.g. mineral oil) on the silica structure while maintaining ideal stability and flow characteristics for the resulting crème. It has been surprisingly found that petrolatum is not difficult to formulate with. Furthermore, while petrolatum provides occlusive benefits it has been found not to interfere with the hair lightening process. Additionally, although it contains petrolatum, the crème formulations can be easily washed out from hair.

In an embodiment, the crème composition comprises:
about 5 to about 60% by weight of a non-volatile oil component,
about 3 to about 30% by weight petrolatum,
about 0.05 to about 5% by weight fumed silica,
about 1 to about 70% by weight of a persulfate salt, and
0% by weight microplastics, wherein all amounts are based on the total weight of the crème composition.

Keratinic fibers or also keratin fibers are understood to mean furs, wool, feathers and, in particular, human hair. Although crème compositions as contemplated herein are primarily suitable for lightening keratin fibers, there is nothing in principle opposed to use in other fields.

Described herein are crème compositions suitable for lightening keratin fibers, in particular human hair, wherein the compositions are free of microplastics yet exhibit desirable rheological properties to be dispensed from a tube or jar and which further exhibit long term stability against phase separation for a shelf life of three years or more in a sealed container. It has been found that using petrolatum and fumed silica in place of block polymers results in a crème composition free of microplastics while at the same time providing stability against settling and phase separation.

### Microplastics

The compositions disclosed herein are free of microplastics, specifically free of synthetic polymers having a size of less than five millimeters. Synthetic polymers include acrylates, acrylates copolymer, acrylates crosspolymer, butylene, carbomer, dimethicone, ethylene, methacrylate copolymer, methacrylate crosspolymer, methyl methacrylate copolymer, methyl methacrylate crosspolymer, nylon, polyacrylamide, polyacrylate, polypropylene, polyurethane, polyvinyl, propylene copolymer, polypropylene, polyvinylpyrrolidone, styrene copolymer, tetrafluoroethylene, vinyl acetate copolymer, vinylpyrrolidone/vinyl acetate copolymer, poloxamer, polyethylene glycol, polypropylene glycol, polyquaternium, polyesters, polyamides, olefin polymer, olefin block polymers, and the like. As used herein, the term “synthetic polymers” excludes celluloses, modified celluloses, and natural polymers. The term “free of microplastics” means there are no microplastics added to the crème composition, either as a standalone ingredient or combined with other ingredients. Any type of microplastics is excluded from the crème compositions and those specifically excluded are olefin block polymers such as those prepared from ethylene, propylene, butylene, octene, styrene, and the like. Block polymers that are considered microplastics include ethylene-octene copolymer, ethylene/propylene/styrene copolymer, butylene/ethylene/styrene copolymer, and the like, or combinations thereof.

Commercially available block polymers formulated as an oil gel vehicle are also excluded from the crème compositions. Examples include VERSAGELs or SYNERGELs by Penreco or other substitute vendors or manufacturers of similar chemistry. The VERSAGEL series includes the following: VERSAGEL C-series, VERSAGEL M-series, VERSAGEL Pseries, VERSAGEL R-series. The M-series includes, but not limited to, VERSAGEL M (mineral oil and ethylene/propylene/styrene copolymer and butylene/ethylene/styrene copolymer), VERSAGEL ME (hydrogenated polyisobutene and ethylene/propylene/styrene copolymer and butylene/ethylene/styrene copolymer), VERSAGEL ML (alkyl benzoate and ethylene/propylene/styrene copolymer and butylene/ethylene/styrene copolymer), VERSAGEL MP (isopropyl palmitate and ethylene/propylene/styrene copolymer and butylene/ethylene/styrene copolymer), VERSAGEL MC (isohexadecane and ethylene/propylene/styrene copolymer and butylene/ethylene/styrene copolymer), VERSAGEL MD (isododecane and ethylene/propylene/styrene copolymer and butylene/ethylene/styrene copolymer), and VERSAGEL MN (isononyl isononanoate and ethylene/propylene/styrene copolymer and butylene/ethylene/styrene copolymer).

### Petrolatum

The petrolatum (CAS registry [8009-03-8]) is a purified mixture of semisolid saturated hydrocarbons having the general formula CₙH₂ₙ₊₂ obtained from petroleum wherein the hydrocarbons are mainly of branched and unbranched chains although some cyclic alkanes and aromatic molecules with paraffin side chains may also be present. The petrolatum material may contain a stabilizer (antioxidant). The petrolatum in combination with the non-volatile oil component, persulfate salt, and fumed silica results in a crème formulation exhibiting excellent stability with reduced oil separation, and stable viscosity for the application area.

The petrolatum can be present in the crème composition in an amount of about 3 to about 30% by weight based on the total weight of the crème composition, specifically about 5 to about 20% by weight, more specifically about 7 to about 18% by weight, and yet more specifically about 10 to about 15% by weight based on the total weight of the crème composition.

### Silica

The fumed silica can be present in the crème composition in an amount of about 0.1 to about 3% by weight based on the total weight of the crème composition, specifically about 0.15 to about 2.5% by weight, more specifically about 0.2 to about 2% by weight, and yet more specifically about 0.3 to about 1% by weight based on the total weight of the crème composition.

### Oil Component

The non-volatile oil component can comprise one or more of mineral oil, hydrogenated polyisobutene, alkyl benzoate, isopropyl palmitate, isohexadecane, isododecane, and isononyl isononanoate. In an embodiment, the non-volatile oil component comprises white mineral oil [8012-95-1], a mixture of refined liquid saturated aliphatic (C₁₄-C₁₈) and cyclic hydrocarbons obtained from petroleum.

The non-volatile oil component can include a cosmetic oil. Non-volatile oils are understood to mean those oils which have a vapor pressure of less than 2.66 Pa (0.02 mm Hg) at 20° C. and an ambient pressure of 1013 hPa.

Still further suitable cosmetic oils as contemplated herein are esters of linear or branched saturated or unsaturated fatty alcohols having 2 to 30 carbon atoms with linear or branched saturated or unsaturated fatty acids having 2 to 30 carbon atoms which may be hydroxylated. Examples include esters of linear or branched saturated fatty alcohols having 2 to 5 carbon atoms with linear or branched saturated or unsaturated fatty acids having 10 to 18 carbon atoms which may be hydroxylated. Further examples thereof are isopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate, 2-ethylhexyl stearate, isopropyl isostearate, isopropyl oleate, isooctylstearate, isononyl stearate, isocetyl stearate, isononyl isononanoate, isotridecyl isononanoate, cetearyl isononanoate, 2-ethylhexyllaurate, 2-ethylhexyl isostearates, 2-ethylhexyl cocoate, 2-octyldodecyl palmitate, butyl octanoic acid 2-butyloctanol, diisotridecylacetate, n-butyl stearate, n-hexyl laurate, n-decyl oleate, oleyl oleate, oleyl erucate, erucyl oleate, erucyl erucate, ethylene glycol diolate, ethylene glycol dipalmitate, n-hexyl laurate, n-decyl oleate, oleyl oleate, oleyl erucate, erucyl oleate, C₁₂-C₁₅ alkyl lactate and di-C₁₂-C₁₃-alkyl malate and the benzoic acid esters of linear or branched C₈-C₂₂-alkanols. Additional examples include benzoic acid C₁₂-Cis-alkyl esters, e.g., commercially available as Finsolv® TN (C₁₂-C₁₅-alkylbenzoate), and benzoic acid isostearyl esters, e.g., available as Finsolv® SB, 2-ethylhexylbenzoate, e.g., available as Finsolv® EB, and benzoic acid 2-octyldodecyl esters, for example, available as Finsolv®BOD.

Further suitable non-volatile oils include the triglycerides of linear or branched, saturated or unsaturated, optionally hydroxylated C₈₋₃₀ fatty acids, in so far as that these are liquid under standard conditions. Natural oils may be used, e.g. soybean oil, cottonseed oil, sunflower oil, palm oil, palm kernel oil, linseed oil, almond oil, castor oil, corn oil, rapeseed oil, olive oil, sesame oil, thistle oil, wheat germ oil, peach kernel oil and the liquid fractions of coconut oil and the like. Also suitable are synthetic triglyceride oils, including capric/caprylic triglycerides, e.g., the commercial products Myritol® 318 or Myritol® 331 (BASF/Cognis) with unbranched fatty acid residues as well as glyceryltriisostearin and glyceryltri (2-ethylhexanoate) with branched fatty acid residues.

Further exemplary non-volatile non-silicone oils are the dicarboxylic acid esters of linear or branched C₂-C₁₀-alkanals, e.g., diisopropylpropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Still further exemplary non-volatile non-silicone oils are the symmetrical, unsymmetrical or cyclic esters of carbonic acid with C₆-C₂₀ alcohols, e.g., di-n-caprylyl carbonate (Cetiol® CC) or di-(2-ethylhexyl) carbonate (Tegosoft DEC). Esters of carbonic acid with C₁-C₅-alcohols, e.g., glycerol carbonate or propylene carbonate, are not compounds suitable as cosmetic oil.

Other exemplary oils include the esters of dimers of unsaturated C₁₂-C₂₂ fatty acids (dimer fatty acids) with monovalent linear, branched or cyclic C₂-C₁₈ alkanals or with polybasic linear or branched C₂-C₆ alkanals.

Other exemplary oils include non-volatile silicone oils. Non-volatile silicone oils as contemplated herein are selected from linear polyalkylsiloxanes having a kinematic viscosity at 25° C. of at least about 5 cSt to about 2000 cSt, in particular linear polydimethylsiloxanes having a kinematic viscosity at 25° C. of from about 5 cSt to about 2000 cSt, specifically from about 10 to about 350 cSt, more specifically from about 50-about 100 cSt, as are available, e.g., under the trade names Dow Corning® 200 or Xiameter PMX, respectively, from Dow Corning and Xiameter. Further non-volatile silicone oils are phenyltrimethicones having a kinematic viscosity at 25° C. of from about 10 to about 100 cSt, specifically from about 15-about 30 cSt, and cetyl dimethicones.

Of the oils mentioned, some have proven to be particularly suitable since they provide physical and chemical stability of the bleaching agents crèmes over long periods of time and are very compatible with the other ingredients as contemplated herein. Highly suitable oils for the non-volatile oil component include mineral oil (also referred to herein as paraffin oil), polyisobutene, hydrogenated polyisobutene, alkylbenzoates, isopropyl palmitate, C₁₄-C₂₂-alkanes, isohexadecane, isododecane, and isononyl isononanoate. The oils can be used singularly or in mixtures of two or more. In an embodiment, the non-volatile oil component is white mineral oil.

The non-volatile oil component can be present in the crème composition in an amount of about 5 to about 60% by weight based on the total weight of the crème composition, specifically about 10 to about 50% by weight, more specifically about 20 to about 40% by weight, and yet more specifically about 22 to about 30% by weight based on the total weight of the crème composition. In an embodiment, the non-volatile oil component is liquid at 25° C.

### Persulfate Salts

The persulfate (alternatively referred to herein as peroxodisulfate or peroxydisulfate) salt can include one or more alkaline earth metal, alkali metal, or ammonium persulfate compounds that exhibit oxidizing activity (generating active oxygen) when combined with the aqueous oxidizing agent composition. Examples of alkali metal persulfates include lithium, sodium, potassium, cesium, and the like. Examples of suitable alkaline earth metals include magnesium, calcium, and the like. In an embodiment, the persulfate salt comprises one or more of sodium persulfate, potassium persulfate, ammonium persulfate, specifically one or more of sodium persulfate and potassium persulfate. The persulfate can be in particulate form, having particle sizes ranging from about 0.1 to 200 micrometers.

The crème compositions can contain the persulfate salt in an amount of about 1 to about 70% by weight based on the total weight of the crème composition. Additional crème composition embodiments contain about 2.5 to about 65% by weight, specifically about 5 to about 60% by weight, more specifically about 7.5 to about 55% by weight, yet more specifically about 10 to about 50% by weight, and still more specifically about 20 to about 40% by weight persulfate based on the total weight of the crème composition.

In certain embodiments, the amount of potassium persulfate is greater than the amount of sodium and/or ammonium persulfate which may be used. Within these embodiments, the weight ratio of potassium persulfate to sodium and/or ammonium persulfate is >about 1.1, specifically >about 1.2, more specifically >about 1.3, and yet more specifically >about 1.4. This weight ratio is determined by dividing the percentage by weight of the amount of potassium persulfate by the sum of the percentage by weight of the amounts of sodium and ammonium persulfate.

The crème composition may contain potassium persulfate and further comprise about 0 to about ≤2.5% by weight, specifically about 0 to about ≤1% by weight, more specifically about 0 to about <0.5% by weight, yet more specifically about 0 to about <0.1%, and still yet more specifically 0% by weight sodium persulfate and/or ammonium persulfate.

The use of the petrolatum and fumed silica provide a stable and homogeneous mixture without separation of the mineral oil and the persulfates, thereby providing a very uniform and homogeneous application during the bleaching of human head hair.

### Additional Ingredients

The crème composition may further comprise one or more additional ingredients includeing, but not limited to, an alkalizer, a benefit agent, a chelating agent, a colorant, an emulsifier, a gelling agent, a humectant, a keratin protein, and a rheology modifier.

The crème composition may contain an alkalizer (also referred to as an alkalizing agent). Suitable alkalizing agents are, for example, ammonia, alkanolamines, basic amino acids, as well as inorganic alkalizing agents such as (earth) alkali metal hydroxides, (earth) alkali metal metasilicates, (earth) alkali metal phosphates and (earth) alkali metal hydrogen phosphates. Exemplary metal ions are lithium, sodium and/or potassium.

Exemplary inorganic alkalizing agents which can be used are sodium hydroxide, potassium hydroxide, calcium hydroxide, barium hydroxide, sodium phosphate, potassium phosphate, sodium silicate, potassium silicate, magnesium silicate, sodium metasilicate, potassium metasilicate, sodium carbonate and potassium carbonate.

In an embodiment, the alkalizer is a sodium silicate and/or a sodium metasilicate.

Further exemplary alkalizing agents which can be used as contemplated herein include alkanolamines from primary, secondary or tertiary amines with a C₂-C₆ alkyl basic body which carries at least one hydroxyl group. Such alkanolamines include 2-aminoethan-1-ol (monoethanolamine), 3-aminopropan-1-ol, 4-aminobutan-1-ol, 5-aminopentan-1-ol, 1-aminopentan-2-ol, (monoisopropanolamine), 1-aminobutane-2-01, 1-aminopentan-2-ol, 1-aminopentan-3-ol, 1-aminopentan-4-ol, 2-amino-2-methylpropanol, 2-amino-2-methylbutanol, 3-amino-2-methylpropan1-ol, 1-amino-2-methylpropan-2-ol, 3-amino-propane-1,2-diol, 2-amino-2-methyl-2-methylpropane-1,3-diol, 2-amino-2-ethyl-1,3-propanediol, N, N-dimethyl ethanolamine, methylglucamine, triethanolamine, diethanolamine and triisopropanolamine.

The basic amino acids which can be used as the alkalinizing agents include L-arginine, D-arginine, D/L-arginine, L-lysine, D-lysine, D/L-lysine, L-ornithine, D-ornithine, D/L-ornithine, L-histidine, D-histidine and/or D/L-histidine.

The crème composition may contain an alkalizer in an amount of about 0.05 to about 25% by weight based on the total weight of the crème composition, specifically about 0.5 to about 20% by weight, more specifically about 1 to about 18% by weight, and yet more specifically about 5 to about 15% by weight based on the total weight of the crème composition.

In an embodiment, the crème composition comprises from 0 to <about 0.1% by weight of ammonia, specifically 0%.

The crème composition may contain a natural polymer as an additional ingredient which may be used as a rheology modifier. Examples are agar agar, carrageenan, alginate, xanthan gum, karaya gum, ghatti gum, tragacanth, scleroglucangum or gum arabic, alginates, pectins, polyoses, guar gums, carob kernel flour, linseed gums, dextrans, starch fractions and derivatives such as amylose, amylopectin and dextrins, gelatin and casein, as well as cellulose derivatives, for example methylcellulose, carboxyalkylcelluloses such as carboxymethylcellulose and hydroxyalkyl celluloses such as hydroxyethyl cellulose.

The crème compositions may further comprise a chelating agent. Suitable chelating agents include, but are not limited to, ethylenediaminetetraacetic acid (EDTA), disodium EDTA, tetrasodium EDTA, cyclodextrin, sodium citrate, tetrasodium pyrophosphate, disodium pyrophosphate, or a combination thereof.

The crème composition can be prepared to have a viscosity suitable for the product category after a short rest period. For example, the crème compositions have a viscosity 24 hours after preparation at 25° C. (Brookfield, Helipath, Spindle TE, 4 rpm, 60 seconds) of from about 50,000 to about 500,000 cps, specifically about 100,000 to about 400,000 cps, and yet more specifically about 200,000 to about 300,000 cps.

The microplastics-free crème compositions prepared with petrolatum are shelf stable, exhibiting no syneresis or separation of the oil component and no hardening within a sealed tube for at least a year, specifically at least two years, and more specifically at least three years.

Also disclosed herein is a method of preparing the crème composition for hair lightening comprising suspending a powder component in an anhydrous gelling composition. The anhydrous gelling composition comprises a non-volatile oil component, petrolatum, and fumed silica as discussed herein. The powder component comprises a persulfate salt, an alkalizer, and additional ingredients such as fillers, rheology modifiers, chelating agents, and the like. The gelling composition provides homogeneous suspension benefits to the powder components. Advantageously, the use of petrolatum in place of a block polymer allows for the preparation of the crème composition at significantly lower processing temperatures, i.e., around 35° C. compared to the higher temperatures >100° C. required when processing block polymer-based formulations. The lower processing temperatures beneficially lower the carbon footprint of these compositions.

In an embodiment, the crème composition comprises≤about 1% by weight free water, specifically a maximum of about 0.5%, of free water. As used herein, “free water” is water which is not contained in the form of water of crystallization, water of hydration or similar molecularly bound water in the composition. The content of water of crystallization, water of hydration or similar molecularly bound water, which is contained in the components used, does not constitute free water in the sense of the present application. Free water is, for example, such water which is added to the composition as a solvent, as a gel activator, or as a solvent component of other active ingredients.

In another embodiment, a method to change the color of keratin fibers comprises application of a crème composition as disclosed herein to the fibers and rinsing the composition off after an exposure time. In a further embodiment, a method to change the color of keratin fibers comprises mixing a crème composition as disclosed herein with an oxidizing agent to form an application mixture, applying the application mixture to the fibers, and rinsing the application mixture off after an exposure time. The oxidizing agent can be hydrogen peroxide. Once mixed with hydrogen peroxide, the application mixture can be applied within a period of a few seconds to one hour, specifically to 30 minutes, and more specifically to 15 minutes. The crème composition or application mixture used in a method for lightening keratin fibers, in particular human hair, can be applied to the keratin-containing fibers, left on the fiber at a temperature of about room temperature to about 45° C. for an exposure time of from about 10 to about 60 minutes, specifically from about 15 to about 50 minutes, and more specifically from about 20 to about 45 minutes, and then rinsed out with water or washed out with a shampoo. During the exposure time of the crème composition or application mixture to the fiber, it may be advantageous to assist the lightening process by supplying heat. The heat supply can be provided by employing an external heat source, such as with the aid of a warm air blower, as well as, particularly in the case of hair lightening in the living subject, by the body temperature of the subject. In the case of the latter possibility, the part to be lightened is usually covered with a hood. An exposure phase at room temperature is also as contemplated herein. The temperature can be between about 20° C. to about 40° C., specifically between about 25° C. to about 38° C., during the exposure time.

After the end of the exposure time, the remaining lightening preparation is rinsed with water or a cleansing agent from the hair. The cleaning agent can be a commercial shampoo and the rinsing process can be carried out with tap water.

The following example are merely illustrative of the crème compositions for hair lightening disclosed herein and are not intended to limit the scope thereof.

### EXAMPLES

### Example 1. Crème Lightener Compositions, Microplastic-Free

A crème composition for hair lightening was prepared with mineral oil, petrolatum, fumed silica, persulfate salts, an alkalizer, and other ingredients, referred to as Formulation 1 in Table 1. Five comparative compositions for hair lightening were also prepared which varied the amount of mineral oil, petrolatum, fumed silica, and two of which contained an amount of a block polymer. Each of Comparative formulations A-E are provided in Table 1.

TABLE 1

| | Formulation | Comparative | Comparative | Comparative | Comparative | Comparative |
|---|---|---|---|---|---|---|
| | 1 | A | B | C | D | E |
| Ingredient | Weight % | | | | | |
| White Mineral Oil | 20-28 | 35-43 | 35-43 | 0 | 0 | 15-20 |
| Petrolatum (8009-03-8) | 10-18 | 0 | 0 | 0 | 35-43 | 0 |
| Fumed Silica | 0.4-0.8 | 0.4-0.8 | 0.8-1.5 | 0.4-0.8 | 0.4-0.8 | 0.4-0.8 |
| VERSAGEL M-500 | 0 | 0 | 0 | 35-43 | 0 | 18-25 |
| (INCI = Mineral Oil | | | | | | |
| (and) Ethylene/Propylene/ | | | | | | |
| Styrene Copolymer | | | | | | |
| (and) Butylene/Ethylene/ | | | | | | |
| Styrene Copolymer) | | | | | | |
| Benefit agent (e.g., | 0-0.3 | 0-0.3 | 0-0.3 | 0-0.3 | 0-0.3 | 0-0.3 |
| Argan oil, Marula oil, | | | | | | |
| shea butter, L-arginine) | | | | | | |
| Persulfate salt (potassium | 25-35 | 25-35 | 25-35 | 25-35 | 25-35 | 25-35 |
| and/or ammonium) | | | | | | |
| Alkalizer (sodium | 12-18 | 12-18 | 12-18 | 12-18 | 12-18 | 12-18 |
| metasilicate and/or | | | | | | |
| sodium silicate) | | | | | | |
| Gelling agent | 2-5 | 2-5 | 2-5 | 2-5 | 2-5 | 2-5 |
| Rheology modifier | 3-5 | 3-5 | 3-5 | 3-5 | 3-5 | 3-5 |
| Emulsifier | 3-5 | 3-5 | 3-5 | 3-5 | 3-5 | 3-5 |
| Humectant | 0.5-1.2 | 0.5-1.2 | 0.5-1.2 | 0.5-1.2 | 0.5-1.2 | 0.5-1.2 |
| Chelating agent | 0.5-1.2 | 0.5-1.2 | 0.5-1.2 | 0.5-1.2 | 0.5-1.2 | 0.5-1.2 |
| Keratin protein | 0-0.06 | 0-0.06 | 0-0.06 | 0-0.06 | 0-0.06 | 0-0.06 |
| Colorant | 0-0.01 | 0-0.01 | 0-0.01 | 0-0.01 | 0-0.01 | 0-0.01 |
| Total | 100 | 100 | 100 | 100 | 100 | 100 |

Formulation 1 (petrolatum+mineral oil+0.4-0.8% fumed silica) contained mineral oil, fumed silica, and petrolatum, but no block polymers, and thus no microplastics. Formulation 1 resulted in a microplastic-free crème lightener that was not dry, not sticky, was easy to mix, and exhibited no syneresis. Advantageously, Formulation 1 had a lower heating requirement, around 35° C., compared to the comparative formulations that contained a block polymer, which required temperatures around 110° C.

Comparative A formulation (Mineral Oil+0.4-0.8% fumed Silica) was formulated with the same amount of fumed silica and no block polymers as Example 1 but only contained mineral oil and no petrolatum. This formulation was easy to mix, however resulted in syneresis as the oil component separated from the powder components.

Comparative B formulation (Mineral Oil+0.8-1.5% fumed Silica) was formulated with no block polymers and nearly twice the amount of fumed silica as Example 1, but without petrolatum. Comparative B resulted in a dry, gritty, difficult to mix material.

Comparative C formulation (Block Polymer+0.4-0.8% fumed Silica) contained the same amount of fumed silica as Formulation 1, but no petrolatum, and in place of the mineral oil, VERSAGEL M-500 block polymer was used. VERSAGEL M-500 contains mineral oil, ethylene/propylene/styrene copolymer and butylene/ethylene/styrene copolymer. Comparative C resulted in a sticky, pituitous mass with incomplete mixing of ingredients. Comparative C also required high heat to swell the block polymers and resulted in a formulation containing undesirable microplastics.

Comparative D was formulated to contain the same amount of fumed silica and no block polymers as Example 1, but had replaced the mineral oil completely with petrolatum. This formulation did not mix well and resulted in a hard, artist clay-like material.

Comparative E contained mineral oil and the same amount of fumed silica as Example 1, but instead of petrolatum, it contained VERSAGEL M-500 block polymer. Although the Comparative E formulation was not dry, not sticky, easy to mix, and exhibited no syneresis, it had several drawbacks: it contained microplastics and required high heat to swell the polymers for complete mixing. The high heat results in a larger carbon footprint for the preparation of such formulations compared to Formulation 1.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A crème composition for lightening keratin fibers, comprising:
about 5 to about 60% by weight of a non-volatile oil component,
about 3 to about 30% by weight petrolatum,
about 0.05 to about 5% by weight fumed silica,
about 1 to about 70% by weight of a persulfate salt, and
0% by weight microplastics,
wherein all amounts are based on the total weight of the crème composition.

2. The composition of claim 1, comprising about 5 to about 20% by weight petrolatum.

3. The composition of claim 1, comprising about 10 to about 15% by weight petrolatum.

4. The composition of claim 1, comprising about 0.2 to about 2% by weight fumed silica.

5. The composition of claim 1, comprising about 0.3 to about 1% by weight fumed silica.

6. The composition of claim 1, wherein the non-volatile oil component is C₁₄-C₂₂-alkanes.

7. The composition of claim 1, wherein the non-volatile oil component is mineral oil, polyisobutene, an alkylbenzoate, isopropyl palmitate, C₁₄-C₂₂-alkanes, isononyl isononanoate, or a combination thereof.

8. The composition of claim 1, wherein the non-volatile oil component is mineral oil.

9. The composition of claim 1, comprising about 10 to about 50% by weight non-volatile oil component.

10. The composition of claim 1, comprising about 20 to about 40% by weight non-volatile oil component.

11. The composition of claim 1, wherein the persulfate salt is one or more of sodium persulfate, potassium persulfate, and ammonium persulfate.

12. The composition of claim 1, wherein the persulfate salt is potassium persulfate and/or ammonium persulfate.

13. The composition of claim 1, comprising about 5 to about 60% by weight of the persulfate salt.

14. The composition of claim 1, comprising about 10 to about 50% by weight of the persulfate salt.

15. The composition of claim 1, comprising about 20 to about 40% by weight of the persulfate salt.

16. The composition of claim 1, further comprising one or more of an alkalizer, a benefit agent, a chelating agent, a colorant, an emulsifier, a gelling agent, a humectant, a keratin protein, and a rheology modifier.
